(19) United States
(12) Patent Application Publication
(10) Pub. No.: US 2024/0423913 A1
(43) Pub. Date: Dec. 26, 2024

(54) TREATMENT USING THERMOSENSITIVE LIPOSOMES

(71) Applicant: Thermosome GmbH, Planegg (DE)

(72) Inventor: Lars Hartwin Lindner, München (DE)

(73) Assignee: Thermosome GmbH, Planegg (DE)

(21) Appl. No.: 18/294,183

(22) PCT Filed: Aug. 16, 2022
(86) PCT No.: PCT/EP2022/072844

§ 371 Date: Feb. 1, 2024

(30) Foreign Application Priority Data
Aug. 18, 2021 (EP) 21191825.5

(51) Int. Cl. A61K 9/127; A61K 31/496; A61K 31/704; A61K 45/06; A61P 35/00

(52) U.S. Cl. CPC A61K 9/127; A61K 31/496; A61K 31/704; A61K 45/06; A61P 35/00

(57) ABSTRACT

The present invention relates to the treatment of cancer by administering thermosensitive liposomes comprising doxorubicin. The liposome administration may preferably be combined with hyperthermia before, during and/or after the liposome administration.

### FIELD OF INVENTION

The present invention relates to the treatment of cancer by administering thermosensitive liposomes comprising doxorubicin. The liposome administration may preferably be combined with hyperthermia before, during and/or after the liposome administration.

### BACKGROUND

Doxorubicin (DOX) is a cytotoxic anthracycline most commonly used to treat cancers of the bladder, breast, stomach, lung, ovaries, thyroid, soft tissue and bone sarcoma, multiple myeloma, and Hodgkin's lymphoma (Rivankar et. al 2014). Further anthracyclines presently employed in cancer treatment include epirubicin, idarubicin, daunorubicin and mitoxantron However, the drug is associated with myelosuppression, mucositis, and arrhythmias as major acute side effects, and cardiomyopathy as a major chronic side effect, which prevents from dosing above a certain cumulative dose limit. To change the pharmacological distribution of the drug, several approaches using different carrier systems have been pursued, which also resulted in reduced drug levels in the heart and other organs (Waterhouse et al. 2001). Furthermore, dexrazoxane is administered as cardioprotectant.

Pegylated-liposomal DOX (such as Doxil®/Caelyx®) are similar effective as non-liposomal DOX but characterized by a lower cardiotoxicity but cause dose-limiting palmar-plantar erythrodysesthesia known as hand-foot syndrome (HFS) characterized by skin eruptions on the palms of the hand and/or soles of the feet, leading to interruption in therapy and decrease in subsequent dosage. A non-pegylated liposomal formulation of DOX (Myocet®) was developed and approved for metastasized breast cancer. It is also characterized by decreased cardiotoxicity and does not show HFS.

For the treatment of soft tissue sarcoma, it could be shown that the addition of regional hyperthermia (HT) to neoadjuvant and adjuvant chemotherapy of 50 mg/m² non-liposomal DOX in combination with etoposide and ifosfamide resulted in increased survival when DOX was administered either as bolus (5 min) or by infusion over 30 min prior to HT (Issels et. al 2018).

To increase the release of the drug in the tumor volume, thermosensitive liposomal (TSL) formulations of DOX which release the drug upon heating of the tumor were evaluated as drug carriers.

A lysolipid-based thermosensitive liposomes (LTSL) formulation developed by Needham et al. (Needham et al. 2000) was evaluated in human clinical trials. This formulation (Thermodox®) is composed of DPPC, 1-stearoyl-sn-glycero-3-phosphocholine (S-Lyso-PC) and 1,2-distearoyl-sn-glycero-3-phosphoethanolamine-N-methoxy (PEG)-2000 (DSPE-PEG2000). In clinical trials, Thermodox® was administered at a DOX dose of 50 mg/m². HT treatment was applied after the administration of the liposomes for the treatment of recurrent breast cancer as disclosed by Zagar et al. (Zagar et al. 2014) or the treatment of liver tumors (Lyon et al. 2018). In a phase III trial for the treatment of unresectable hepatocellular carcinoma, the HT treatment was initiated at least 15 minutes after start of administration of the DOX LTSL and was completed within 3 hours after infusion initiation. However, no increase in progression free survival or overall survival in the overall study population was observed in the investigated schedule (Tak et al. 2018).

Further to the lysolipid-based thermosensitive liposomes (LTSL) formulation, a thermosensitive liposomal formulation based on the phospholipid class of 1,2-diacyl-sn-glycero-3-phospho-rac-oligo-glycerol (PGn) for DOX was developed to overcome stability issues of LTSLs (Lindner et al. 2004). A specific composition comprising DPPC/DSPC/DPPG₂ 50:20:30 (mol/mol) (DPPG₂-TSL_{30%}) with encapsulated DOX was described by Hossann et al. (Hossann et al. 2007).

Until to date, DPPG₂-TSL_{30%} formulations were investigated in preclinical models. Willerding and colleagues investigated DPPG₂-TSL_{30%} formulations in a soft tissue sarcoma rat model. Due to the experimental animal setting, hyperthermia was induced with cold light lamp and a water bath. Liposomes were administered at a maximum DOX dose of 2 mg/kg at a tumor temperature of 40° C. HT was continued for 60 min after bolus injection of liposomes (Willerding et al. 2016). In a study by Hossann et al. (Hossann et al. 2021) DPPG₂-TSL_{30%} DOX was investigated in cats with locally advanced spontaneous fibrosarcomas (soft tissue sarcoma) at a maximum dose of 1.0 mg/kg in combination with HT induced by means of a superficial radiofrequency applicator. HT with a target temperature of 41.5 C was applied over 60 min simultaneously to liposomal DOX administration in the first 15 min of HT application by infusion. In a study by van Valenberg et al. (van Valenberg et al. 2021) DOX distribution in the bladder wall after DPPG₂-TSL_{30%} infusion and application of hyperthermia by a urinary catheter was investigated in a pig model. Here, liposomal DOX was administered as soon as the intrevesical temperature was stable at 43° C. This temperature was aimed to be maintained in the bladder for 60 or 90 minutes and DOX-uptake into the bladder wall (mucosa, detrusor, serosa) was finally determined. However, in contrast to the previously described sarcoma animal model studies, no doxorubicin uptake into tumor tissue was investigated. Notably, superficial hyperthermia was used only to heat bladder wall, which is a few millimeters thick, in contrast to soft tissue sarcoma which would require heating and DOX-uptake in significantly larger tissue volumes.

No administration schedule for DPPG₂-TSL formulations in combination with HT is presently available for the human patient.

### Problem Underlying the Invention

In view of the prior art, it was the general problem underlying the present invention to provide an administration schedule for TSL formulations with encapsulated DOX for the human patient.

### DESCRIPTION OF THE INVENTION

The problem is solved by the liposome and the methods according to the claims. Further embodiments of the invention are outlined throughout the description.

In a first aspect, the invention relates to a thermosensitive liposome comprising doxorubicin or a pharmaceutically acceptable salt or derivative thereof for use in a method of treating cancer in a human subject encapsulated in the aqueous core of the liposome, wherein the liposome is administered to the subject at a dose of about 20 to about 80 mg/m².

In a further aspect the present invention relates to a method of treating cancer in a human subject, comprising the step of administering a liposome comprising doxorubicin (DOX) or a pharmaceutically acceptable salt or derivative thereof at a dose of about 20 mg/m² to about 80 mg/m².

As used in this specification and the appended claims, the singular forms “a,” “an” and “the” include plural references unless the context clearly dictates otherwise. For example, according to the invention non only one single liposome but a plurality of liposomes that together comprise the respective dose of doxorubicin will be administered.

“About” in the context of amount values refers to an average deviation of maximum +/−20%, preferably +/−10%, most preferably +/−5% based on the indicated value. For example, an amount of about 20 mg/m² refers to 20 mg/m²+/−6 mg/m², preferably 20 mg/m²+/−4 mg/m², most preferably 20 mg/m²+/−2 mg/m². This includes also the value itself with any deviation.

The term “derivative” refers to a compound derived from some other compound while maintaining its general structural features. Derivatives may be obtained for example by chemical functionalization or derivatization such as epirubicin and pirarubicin.

Within the context of the present invention, the term “cancer” refers to a class of diseases where cells undergo uncontrolled growth with the potential to become malignant through the acquisition of various aberrant during their development.

Within the context of the present invention, a dose in mg/m² refers to a dose of the recited amounts in milligram of doxorubicin or a pharmaceutically acceptable salt or derivative thereof per square meter of the subject's body surface. The respective dose may easily be converted into milligram per kilogram (mg/kg) of subject's body mass. Accordingly, the present invention encompasses doses in mg/kg which are equivalent to the recited doses in mg/m². For the human subject, doses may be easily converted based on the relation of body surface to body height and height for example S=M^{0.425}×H^{0.725}×71.84, wherein S=body surface, M=body mass, H=body height.

As an example, the liposome may comprise 1,2-dipalmitoyl-sn-glycero-3-phosphocholine (DPPC), 1-stearoyl-sn-glycero-3-phosphocholine (S-Lyso-PC) and 1,2-distearoyl-sn-glycero-3-phosphoethanolamine-N-methoxy (PEG)-2000 (DSPE-PEG2000).

However, in a preferred embodiment, the liposome does not comprise a lysolipid in the bilayer. Lysolipids are lipids comprising a single carbon chain and a polar head group. The lysolipid may be a lysosphingolipids or a lysoglycerophospholipids.

In the highly preferred embodiment, the liposome comprises 1,2-dipalmitoyl-sn-glycero-3-phosphooligoglycerol (DPPGₙ) in the bilayer, wherein n is preferably 2 to 4. Most preferably the liposome comprises 1,2-dipalmitoyl-sn-glycero-3-phosphooligodiglycerol (DPPG₂), in the bilayer.

The dose may be from about 20 to about 80 mg/m², preferably from about 30 to about 75 mg/m², from about 35 to about 70 mg/m², from about 40 to about 65 mg/m², from about 45 to about 65 mg/m², from about 45 to about 65 mg/m², or from about 40 to about 60 mg/m². More preferably, the dose may be from about 45 to about 60 mg/m² or from 45 to about 55 mg/m², most preferably from about 50 to about 60 mg/m².

Within the dose ranges disclosed above, the dose may be at least 25 mg/m², 30 mg/m², 35 mg/m², 40 mg/m², or 45 mg/m². Furthermore, the dose may be no more than 70 mg/m², 65 mg/m², 60 mg/m², or 55 mg/m² within the dose ranges disclosed above.

Lipid bilayers can exist in a liquid phase (liquid crystalline phase), which is characterized by a high fluidity, or in a solid phase (gel phase, solid gel phase), which is characterized by a low fluidity. The phase in which a bilayer with a specific composition exists in a specific environment is mainly determined by the temperature and the identity of the molecules comprised in said bilayer. In the solid phase, the lipids comprised in said bilayer are ordered in a structured way reminiscent of a crystalline structure, whereas in the liquid phase the lipids are unordered and are able to diffuse freely in the plane of the bilayer.

A bilayer with a specific composition, e.g., consisting of a specific set of lipids in specific molar ratios, will exist in the solid phase if the temperature is lower than the gel to liquid phase transition temperature, and will exist in the liquid phase if the temperature is higher than the gel to liquid phase transition temperature. In the context of this application, the gel to liquid phase transition temperature is also called the transition temperature or Tₘ. The transition temperature is mainly determined by the identity of the molecules comprised in said bilayer, and hence by the lipids comprised in said bilayer. At a temperature around the gel to liquid phase transition temperature, the structure of a bilayer comprises both freely diffusing lipids and rafts of ordered lipids.

The Tₘ of thermosensitive liposomes, especially TSL comprising 1,2-dipalmitoyl-sn-glycero-3-phosphodiglycerol (DPPG₂), is preferably in the range from about 38° C. to about 45° C., preferably from about 41° C. to about 43° C.

Due to the change of lipid bilayer structure around the transition temperature, the lipid bilayer becomes permeable for active agents, such as doxorubicin comprised in the intraliposomal buffer or the lipid bilayer. This increased permeability results in a release of the active agent comprise in the liposome into the extraliposomal buffer, or extraliposomal physiological liquid, such as blood. Generally, it is not required according to the invention that 100% of the active agent comprised in the liposome is released at or above Tₘ. The active agent, such as doxorubicin or a pharmaceutically acceptable salt or derivative thereof, released from the liposome may then exert its pharmacological action to treat a disease.

Release of the active agent from thermosensitive liposomes according to the invention after the administered of the liposomes to a subject's body is generally achieved by administering a heat treatment to the subject's body, more preferably to a part of the subject's body. In case a heat treatment is administered only to a part of the subject's body, the heat treatment will be administered to the part of the body where the active agent is desired to exert its pharmacological action, thus in a method of treating cancer the part of the body where a cancer lesion is located.

A “heat treatment” according to the present invention refers to the administration of energy to the human body or parts thereof which results in an increase of the temperature of the human body or parts thereof above the overall mean body temperature of a healthy human subject. Within the context of this invention, the overall mean oral temperature for a healthy human aged 18 to 40 years is 36.8.+/−0.0.4° C. An admiration of a heat treatment in the sense of the present invention is common known as administration of “hyperthermia” in the art. Thus, the term “heat treatment” and “hyperthermia” or the abbreviation “HT” may be used interchangeably herein.

When thermosensitive liposomes, especially thermosensitive liposomes comprising 1,2-dipalmitoyl-sn-glycero-3-phosphodiglycerol (DPPG₂) comprising doxorubicin, a doxorubicin derivative or said pharmaceutically acceptable salt thereof are administered to a human subject and an additional heat treatment is administered to the subject, especially to an area of the human body which comprises a tumor, the increase of body temperature and thereby tumor temperature to or above the Tₘ of the liposome results in a release of the doxorubicin from the liposome.

Therefore, in an important aspect of the present invention, a heat treatment is administered to the subject to be treated prior to the administration of the liposome to the subject until a tumor temperature in the range from at least about 3° C. lower than Tₘ to at least Tₘ of administered liposome is reached. Preferably, a heat treatment is administered until a tumor temperature in a range of from about 38° C. to about 45° C., or from about 40° C. to about 45° C., preferably from about 41° C. to about 43° C., most preferably from about 41.5° C. to about 43° C. is reached. Within the afore mentioned temperature range, a tumor temperature of at least about 38.5° C., least about 39° C., least about 39.5° C., least about 40° C., least about 40.5° C., least about 41° C., least about 41.5° C. or at least about 42° C. may be reached. With the afore mentioned temperature range a tumor temperature of up to about 44.5° C., up to about 44° C., up to about 43.5° C., up to about 43° C., up to about 42.5° C. or up to about 42° C. may be reached. Furthermore, the administration of a heat treatment prior to the administration of the liposomes until a specific tumor temperature is reached encompasses embodiments wherein the respective heat treatment is maintained at least until the administration of liposomes to the subject is started to maintain the desired tumor temperature.

In one embodiment of the invention, the administration of the liposome may be started when a tumor temperature in a range of from about 38° C. to about 45° C., or from about 40° C. to about 45° C., preferably from about 41° C. to about 43° C., most preferably from about 41.5° C. to about 43° C. is reached. Within the afore mentioned temperature range, the administration of the liposome may be started when a tumor temperature of at least about 38.5° C., least about 39° C., least about 39.5° C., least about 40° C., least about 40.5° C., least about 41° C., least about 41.5° C. or at least about 42° C. is reached.

A heat treatment already prior to the administered of the liposome may facilitate a fast and efficient releases of the active agent to the tumor.

Preferably the heat administered prior to the administration of the liposome may be administered for a time period of at least about 10 min, at least about 20 min, or at least about 25 min prior to the administration of the liposome. Preferably the heat treatment is administered for a time period of about 30 min prior to the administration of the liposome.

In a further preferred aspect of the present invention, a heat treatment is administered to the subject to be treated during the administration of the liposome to the subject which increases the tumor temperature or maintains the tumor temperature to/at a temperature in a range from about 38° C. to about 45° C., from about 40.0° C. to about 45° C., preferably from about 41.0° C. to about 43° C., or most preferably from about 41.5° C. to about 43° C. is reached. When a heat treatment is administered during the administration of the liposome, it is understood that the heat treatment is administered concomitant to the administration of the liposome. Within the afore mentioned temperature range, the tumor temperature may be increased to, or maintained at a temperature of least about 38.5° C., least about 39° C., least about 39.5° C., least about 40° C., least about 40.5° C., least about 41° C., least about 41.5° C. or at least about 42° C. Within the afore mentioned temperature range, the tumor temperature may be increased to, or maintained at a temperature of up to about 44.5° C., up to about 44° C., up to about 43.5° C., up to about 43° C., up to about 42.5° C. or up to about 42° C.

In an alternative preferred embodiment, the administration of the heat treatment is started after or concomitantly with the start of the administration of the liposome. The administration of the heat treatment may be started at up to about 5 min, up to about 10 min, up to about 15 min, up to about 20 min, up to about 30 min, or up to about 45 min after the start of the administration of the liposome. The administration of the heat treatment may be started at least about 5 min, at least about 10 min, at least about 15 min, at least about 20 min, at least about 30 min, or at least about 45 min after the start of the administration of the liposome.

In an embodiment wherein a heat treatment is administered already prior to the administration of the liposomes, the heat treatment is preferably administered to the subject also during the administration of the liposome to maintain a desired tumor temperature. When a tumor temperature within a given range is “maintained” according to the present invention, the temperature may vary within this range over time and may also sometimes be outside of this this range. Nevertheless, the temperature will be considered as being maintained according to the present invention. Also, in an embodiment wherein a heat treatment is administered to the subject during the administration of the liposome to maintain a specific tumor temperature, the heat treatment may be terminated prior to the end of the liposome administration so that the temperature is not maintained in a given temperature range over the entire period of liposome administration.

However, in a preferred embodiment of the present invention, the heat treatment is administered over the entire period of the liposome administration to maintain a tumor temperature in a range described above over the entire period of the liposome administration.

In a further embodiment of the invention, the heat treatment may be continued for a period of time also until after the administration of the liposome has been terminated to maintain a tumor temperature in the above-described temperature range. Thus, the heat treatment may be administered after the administration of the liposome is terminated to maintain a tumor temperature in a temperature range from about 38° C. to about 45° C., or from about 40.0° C. to about 45° C., preferably from about 41.0° C. to about 43° C. is reached. Within the afore mentioned temperature range, a temperature of least about 39° C., least about 39.5° C., least about 40° C., least about 40.5° C., least about 41° C., least about 41.5° C. or at least about 42° C. may be maintained. Within the afore mentioned temperature range, a tumor temperature of up to about 44.5° C., up to about 44° C., up to about 43.5° C., up to about 43° C., up to about 42.5° C. or up to about 42° C. may be maintained.

The administration of the heat treatment may for example be continued until at least about 10 min, at least about 20, at least about 30 min after termination of the administration of the liposome. The administration of the heat treatment may for example be continued up to about 90 min, up to about 75 min, up to about 60 min, preferably until up to about 45 min, most preferably until up to 30 min after termination of the administration of the liposome.

In a highly preferred embodiment, the heat treatment is administered prior to the administration of the liposome, administered over the entire period of the liposome administration, and continued until after termination of the administration of the liposome. Length and intensity of the heat treatment in the different phases is preferably carried out within the ranges disclosed above.

The tumor temperature may be determined minimally invasive by thermal probes and a means for inserting and removing such probes into the tumor tissue or to the close vicinity of the tumor tissue. The construction of thermal probes is known to those skilled in the art, and suitable thermal probes include, for example, miniature thermistors and wire thermocouples. For example, closed-tip hyperthermia catheters may be used. Preferably probes which do not interfere with a high frequency electromagnetic field are used, for example Bowman probes. Insertion of the thermal probes may preferably be controlled by suitable imaging technologies, for example computer tomography or magnetic resonance imaging. The use of CT fluoroscopy-guided closed-tip hyperthermia catheters for determining tumor temperature is disclosed by Strobl et al. (Strobl et al. 2016).

Alternatively, non-invasive techniques such as magnetic resonance thermometry as disclosed by Carter et al. (Carter et al. 1998) may be used to determine the tumor temperature during the heat treatment.

According to the present invention, the liposomes are typically administered systemically by parenteral such as intravenous administration. Preferably the liposomes are administered intravenously. Prior to administration a composition comprising the thermosensitive liposome may be diluted in a suitable clinical infusion carrier, such as for example 0.9% NaCl and/or 5% glucose.

The cancer treated according to the present invention may preferably be a locally advanced or metastatic cancer. Locally advanced means that the cancer has grown outside of the tissue it originated from but within the body part it originates from. Thus, the cancer has not yet disseminated to other parts of the body. In metastatic cancers, the cancer has disseminated from its tissue and body part of origin to other parts of the body.

In a highly preferred aspect of the invention, the treated cancer is a soft tissue sarcoma (STS). STS are a group of cancers that originated in the tissues that connect, support and surround other body structures, such as muscle, fat, blood vessels, nerves, tendons and the lining of joints.

Preferably, the STS may be an undifferentiated pleomorphic sarcoma (UPS), liposarcoma, for example differentiated liposarcoma, dedifferentiated liposarcoma, myxoid liposarcoma, pleomorphic liposarcoma; leiomyosarcoma, synovial sarcoma, angiosarcoma, epithelioid sarcoma, malignant peripheral nerve sheath tumor (MPNST), rhabdomyosarcoma, for example alveolar rhabdomyosarcoma, embryonal rhabdomyosarcoma, pleomorphic rhabdomyosarcoma; solitary fibrous tumor, myxofibrosarcoma, fibrosarcoma, uterine sarcoma, for example uterine leiomyosarcoma, endometrial stromal sarcoma; desmoplastic small round cell sarcoma (DSRCT), desmoids, or kaposi sarcoma.

The cancer may also be a bone sarcoma, and most preferably Ewing sarcoma, osteosarcoma or chondrosarcoma.

The heat treatment according to the present invention may generally be administered in form of local hyperthermia, e.g. superficial local hyperthermia, locoregional, and/or regional hyperthermia, preferably as regional deep hyperthermia. Superficial hyperthermia induced by one applicator typically has a penetration depth from the skin surface of about 2 to about 4 cm at a frequency of about 400 to about 1000 MHZ, but may be increased to up to about 10 cm at a frequency of about 10 MHZ. Generally, the choice of hyperthermia treatment will depend on the tumor type to be treated and the specific condition in the patient to be treated.

Local hyperthermia may especially be used for relatively small tumors (≤3 cm up to 5-6 cm) located superficially or within an available body cavity such as the rectum or esophagus. In local hyperthermia, heat is applied to a small area restricted to the tumor and directly adjacent tissue. Typically, local superficial hyperthermia has a heat focus for the increased temperature range as disclosed above of about 200 to about 400 cm³ when one applicator is used, as disclosed by Paulides et al. (Paulides et al. 2020). Local deep hyperthermia typically has a heat focus of about 15 to about 250 cm³. For external local hyperthermia, different types of energy may be used to apply heat, including microwave, radiofrequency, and ultrasound. External local hyperthermia may be performed with applicators of different kinds, for example waveguide, spiral, or current sheets, placed on the surface of superficial tumors with a contacting medium.

Local intraluminal, endocavitary (also termed intracavitary) or interstitial local hyperthermia may be used to treat tumors within or near body cavities by inserting microwave, radiofrequency, and ultrasound antennas in natural openings of hollow organs or by implanting antennas into the respective tumor or organ. Local interstitial hyperthermia typically has a heat focus from about 30 to about 120 cm³. Local intraluminal/endocavitary hyperthermia has a heat focus from about 5 to about 30 cm³.

In regional or locoregional hyperthermia, moderately large volumes of body parts, such as the thorax or pelvis, including the cancerous region as well as the surrounding healthy tissue are heated. In regional deep hyperthermia, the heat focus for the increased temperature range as disclosed above may typically be in a volume of about 1750 to about 4000 cm³, about 250 to about 1750 cm³ in locoregional deep hyperthermia as disclosed by Paulides et al. (Paulides et al. 2020). Within the scope of the present disclosure the “heat focus” refers to the area of tissue that is heated to a temperature of at least about 40° C. The remainder of the body is kept as close to normal temperature as possible. Regional hyperthermia may for example be administered as regional deep hyperthermia or regional perfusion hyperthermia.

The regional or locoregional hyperthermia according to the invention is especially suitable to treat tumors of medium to larger volumes, having a volume of at least about 30 cm³, preferably at least about 50 cm³, more preferably at least about 100 cm³, even more preferably at least about 150 cm³, most preferably at least about 200 cm³. The tumor treated according to the present invention may have a volume of up to about 34.000 cm³, preferably up to about 20.000 cm³, more preferably 10.000 cm³.

The tumors treated according to the present invention may have a diameter of at least about 4 cm, preferably at least about 5 cm, more preferably at least about 6 cm, even more preferably at least about 8 cm, most preferably at least about 10 cm. The tumor treated according to the present invention may have a diameter of up to about 40 cm, preferably up to about 30 cm, more preferably 15 cm.

The tumor treated may be localized in the trunk, the extremities, visceral/abdominal, and/or retroperitoneal. In a specific embodiment, the tumor treated according to the invention is not a bladder tumor

In a preferred embodiment the heat treatment according to the present invention is administered in form of regional deep or locoregional deep hyperthermia.

Preferably the heat treatment according to the invention is administered in form of electromagnetic-based hyperthermia techniques, for example as radio or microwave frequency induced hyperthermia, ultrasound induced hyperthermia or hyperthermic perfusion.

For regional, locoregional, or local hyperthermia, heat treatment may be administered by external electromagnetic-based hyperthermia techniques such as microwave or radiofrequency. Suitable applicators known to the person in the art are positioned around the body, cavity or organ to be treated, and microwave or radiofrequency energy is focused on an area to raise the temperature. Preferably the ultrasound or microwave frequency induced hyperthermia is administered by external administration, thus by applicators positioned outside around the body. Within the context of the present invention, external administration does not include the use of applicators, such as for example antennas, which are introduced into a body cavity.

The external electromagnetic-based hyperthermia treatment may for example treat tumors which are located in a distance of at least 1 cm, 2 cm, 4 cm, 6 cm, 8 cm, 10 cm, or 20 cm from a body surface. Within this context, the “body surface” includes the internal surface of body cavities such bladder or intestine.

Radio frequency induced hyperthermia may generally be carried out at frequencies of 3 Hz to 300 MHz, whereas microwave induced hyperthermia is carried out at frequencies of 300 MHz to 300 GHz. For local superficial hyperthermia microwave frequencies of about 400 MHz to about 1000 MHz may preferably be used. For regional deep hyperthermia frequencies up to 100 MHz may preferably be used. For deep locoregional hyperthermia frequencies of about 100 MHz to about 300 MHz may preferably be used. Suitable radiation devices and applicators are known in the art and commercially available. For regional deep or locoregional deep hyperthermia the Alba 4D device (Medlogix, Rome, Italy) with Alba 4D applicator; the BSD-500 device (Pyrexar Medical, West Valley City, USA) with MA-151, MA-100, MA-120, SA-308, SA-510, SA-812, SA-248, or MA-251 applicators; the BSD-2000 devices (Pyrexar) with Σ-30, Σ-60, Σ-Ellipse applicators; the BSD-2000 3D device (Pyrexar) with Σ-Eye applicator; the BSD-2000 3D/MR device with E-30-MR, or Σ-Eye-MR applicator, or the THERMOTRON-RF8 (Yamamoto Vinita, Osaka, Japan) device may be used for example. Specifically, for head and neck cancer the HYPERcollar device developed by Drizdal and colleagues may be used (Drizdal et al. 2019). The frequencies applied may generally be in the range from about 60 MHz to about 180 MHZ, about 70 MHz to about 140 MHz for tumors located in the pelvic region and about 434 MHz for head and neck region. The forward power may for example range from 0 to about 3000 watts, preferably from 0 to about 2500 watts, most preferably from 0 to about 2000 watts. Hyperthermia application may be planned and conducted for example as described by Aklan et. (Aklan et al. 2019), Kok et al. (Kok et al. 2015), or described in Example 1. The person skilled in the art will adapt the parameters depending on the specific patient and tumor to be treated. Preferably, regional deep hyperthermia is carried out in accordance with the relevant guidelines by Bruggmoser et al. (Bruggmoser et al. 2012).

Ultrasound induced hyperthermia may be carried out as disclosed in Zhu (2019) and related references.

According to the invention the liposome may be administered in a treatment cycle every 7 to 28 days, preferably every 14 to 25 days more preferably every 20 to 22 days, most preferably every 21 days. The liposome may be administered in a treatment cycle every at least every 7 days, at least every 8 days, at least every 9 days, at least every 10 days, at least every 11 days, at least every 12 days, at least every 13 days, at least every 14 days, at least every 15 days, at least every 16 days, at least every 17 days, at least every 18 days, at least every 19 days, at least every 20 days, at least every 21 days. Within the context of the present invention, a treatment cycle includes the admiration of the liposome and the administration of other therapeutic means or means supporting the therapy as well as carrying out other steps, such as diagnostic steps, related to the treatment if appropriate and/or necessary. An admiration in a treatment cycle every 21 days etc. is understood as an admiration on the 21^{st} day after the last administration.

In a highly preferred embodiment, a heat treatment is administered before and/or during and/or after the administration of the liposome as disclosed above in every treatment cycle.

The liposome according to the invention may be administered for 2 to 10, preferably 4 to 8, more preferably 5 to 7 cycles, and most preferably 6 cycles. The liposome may be administered for at least 2, at least 3, at least 4, at least 5, at least 6, or at least 7 cycles.

Dosing of the doxorubicin comprising liposomes and/or duration and heat treatment may be varied between the different cycles depending on side effects, response or other parameters observed.

In a further preferred embodiment, a premedication, preferably an antiemetic compound and/or a compound that reduces or eliminates hypersensitivity reactions is administered prior to the admiration to the liposome. The compound that reduces or eliminates hypersensitivity reactions may be selected from the group comprising steroids, antihistamines; H1-, H2, and combinations thereof in a sufficient amount to prevent or reduce side effects including fatal anaphylactic reactions. Preferably, the compound is selected from the group comprising steroids, and/or H1-receptor antagonists. Said compound can also be selected from the group comprising ranitidine, dexamethasone, diphenhydramine, famotidine, hydrocortisone, clemastine, cimetidine, prednisolone, chlorpheniramine, chlorphenamine, dimethindene maleate, and promethazine. Dexrazoxane may especially administered as premedication in patients which have received anthracycline before.

The liposome according to the invention has a bilayer comprising 1,2-dipalmitoyl-sn-glycero-3-phosphodiglycerol (DPPG₂) wherein said DPPG₂, is represented by Formula (I):

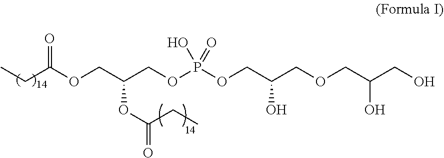

In a preferred embodiment of the invention, the liposome comprises a molar concentration of said DPPG₂ in said bilayer of at least 10 mol %, at least 15 mol %, or at least 20 mol %, or at least 25 mol %. The molar concentration of 1,2-dipalmitoyl-sn-glycero-3-phosphodiglycerol (DPPG₂) in said bilayer may be from about 15 mol % up to about 55 mol %, preferably from about 15 mol % up to about 45 mol %, more preferably from about 25 mol % up to about 35 mol %. Most preferably the molar concentration of 1,2-dipalmitoyl-sn-glycero-3-phosphodiglycerol (DPPG₂) in said bilayer is about 30 mol %.

In a further preferred embodiment of the invention, the liposome comprises DPPG₂ in said bilayer from 26 mol % up to 34 mol %, from 27 mol % up to 33 mol %, from 28 mol % up to 32 mol %, from 29 mol % up to 31 mol %.

According to the invention, the bilayer of the liposome further comprises at least one lipid selected from the group comprising 1,2-dipalmitoyl-sn-glycero-3-phosphatidylcholine (DPPC), 1,2-dipalmitoyl-sn-glycero-3-phosphatidylethanolamine (DPPE), 1,2-distearyl-sn-glycero-3-phosphatidylcholine (DSPC), 1,2-distearyl-sn-glycero-3-phosphatidylethanolamine (DSPE), N-methoxy-(PEG)-2000-1,2-distearyl-sn-glycero-3-phosphatidylethanolamine (DSPE-PEG₂₀₀₀).

Most preferably, the bilayer of the liposome according to the invention comprises or consists of 1,2-dipalmitoyl-sn-glycero-3-phosphatidylcholine (DPPC), 1,2-distearyl-sn-glycero-3-phosphatidylcholine (DSPC), and 1,2-dipalmitoyl-sn-glycero-3-phosphatidyldiglycerol (DPPG₂).

In a specific embodiment the bilayer of the liposome comprises a molar concentration of 1,2-dipalmitoyl-sn-glycero-3-phosphocholine (DPPC) in said bilayer from 30 mol % up to 70 mol %, preferably from 45 mol % up to 55 mol %; and/or a molar concentration of 1,2-distearoyl-sn-glycero-3-phosphocholine (DSPC) in said bilayer from 5 mol % up to 35 mol %, preferably from 15 mol % up to 25 mol %; and/or a molar concentration of 1,2-dipalmitoyl-sn-glycero-3-phosphodiglycerol (DPPG₂) in said bilayer from 15 mol % up to 45 mol %, preferably from 25 mol % up to 35 mol %.

Most preferably the liposome comprises a molar concentration of 1,2-dipalmitoyl-sn-glycero-3-phosphocholine (DPPC) in said bilayer of about 50 mol %, 1,2-distearoyl-sn-glycero-3-phosphocholine (DSPC) in said bilayer of about 20 mol %; and 1,2-dipalmitoyl-sn-glycero-3-phosphodiglycerol (DPPG₂) in said bilayer of about 30 mol %.

The liposome may preferably have a diameter from 100 nanometers up to 150 nanometers measured by dynamic light scattering as z average.

The molar ratio between doxorubicin, a doxorubicin derivative or said pharmaceutically acceptable salt thereof and the lipids comprised in liposome is from 0.06 up to 0.10, preferably from 0.07 up to 0.09 doxorubicin.

The intraliposomal buffer comprised in the liposomes according to the invention has a pH from 6 up to 8, preferably from 7 up to 8, most preferably from 7.2 up to 7.6.

Liposomes according to the invention comprising doxorubicin or a pharmaceutically acceptable salt or derivative thereof may be prepared via a wide range of different techniques such as lipid film hydration, ethanol injection and the extrusion method, as known by the skilled person.

Generally, during the preparation of a liposome comprising doxorubicin or a pharmaceutically acceptable salt or derivative thereof, an unloaded liposome with no active pharmaceutical ingredient in the intraliposomal buffer is prepared first by the afore mentioned methods.

Subsequently, the doxorubicin or a pharmaceutically acceptable salt or derivative thereof is loaded to the liposome by means of a gradient as disclosed by Hossann and colleagues for with a pH gradient (Hossann et al. 2007; Hossann et al. 2010). A further method of preparing the liposomes employed in the present invention is disclosed in patent applications claiming priority to the EP patent application No. 20184542.7.

The liposome according to the present invention is comprised in a pharmaceutical composition usually comprising a pharmaceutically accepted carrier, diluent and/or excipient. In a preferred embodiment, a composition of the current invention comprises a thermosensitive liposome as defined herein and further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy, 20th Edition. Baltimore, MD: Lippincott Williams & Wilkins, 2000. A preferred carrier according to the present invention is water for injection comprising further pharmaceutically accepted excipients, preferably as disclosed in the following. The aqueous carrier wherein the liposomes are comprised is also referred to herein as extraliposomal buffer.

The extraliposomal buffer preferably comprises a cryoprotectant, preferably a disaccharide, for example trehalose or sucrose, most preferably trehalose. Preferably the cryoprotectant is comprised in the extraliposomal buffer in amount from about 5 to about 15% (w/v), more preferably about 10% (w/v).

The extraliposomal buffer may further comprise a salt, preferably Na/K phosphate. Preferably the salt has a concentration from about 5 to about 20 mM, more preferably from about 8 to about 12 mM.

Most preferably, the extraliposomal buffer comprises 10% (w/v) trehalose, 10.5 mM Na/K phosphate. The extraliposomal buffer according to the invention has a pH from 6 up to 8, preferably from 7 up to 8, most preferably from 7.2 up to 7.6.

All of the compounds, compositions, and methods disclosed and claimed herein can be made and carried out without undue experimentation in light of the present disclosure. While the compounds, compositions and methods of this invention have been described in terms of preferred embodiments, it will be apparent to those skilled in the art that variations may be applied to the composition, methods and in the steps described herein without departing from the concept, spirit and scope of the invention. All such variations apparent to those skilled in the art are deemed to be within the spirit, scope and concept of the invention as defined by the appended claims.

### EXAMPLES

### Example 1: Clinical Phase 1 Trial with DPPG₂-TSL-DOX

### 1. Study Population

Subjects are eligible with locally advanced (unresectable) or metastatic sarcoma, aged 18 years and for whom treatment with DOX monotherapy is appropriate.

### II. Inclusion Criteria

1. Age at the time of consent is 18 years.
2. Subject has provided written informed consent prior to any study-specific procedures.
3. Locally advanced (unresectable) or metastatic soft tissue sarcoma (STS) histologically diagnosed by local pathology review for which treatment with DOX monotherapy is appropriate as confirmed by the investigator.
4. Pretreatment with DOX or any DOX combination chemotherapy provided at least stable disease was achieved. For patients who received DOX in an adjuvant setting, local recurrence free interval of >6 months is required.
5. Progressive disease not suitable for surgery after
   a) only one further line of chemotherapy (including tyrosine-kinase inhibitor, TKI) if the regional hyperthermia (RHT) field targets the clinically relevant tumor manifestation/s (e.g. locally advanced or multifocal intraabdominal STS; diffuse metastatic STS in which RHT of a tumor manifestation [e.g. liver] is considered relevant although other systemic metastases are present that do not endanger the subject, as per the judgement of the investigator)
      or
   b) two or more further lines of chemotherapies (including TKI) for subjects with metastatic STS and a tumor manifestation suitable for RHT.
6. All previous oncological treatments must have been completed ≥3 weeks (21 days) prior to the first dose of study treatment, ensuring a sufficient washout period.
7. Measurable disease as defined by the Response Evaluation Criteria in Solid Tumors (RECIST 1.1) (Eisenhauer et al. 2009).
8. Tumor accessible for RHT with Pyrexar BSD-2000® or Pyrexar BSD-500®
9. Left ventricular ejection fraction (LVEF) >50% (within 28 days prior to enrolment).
10. Adequate hematologic, organ and coagulation function within 14 days prior to enrolment as assessed by local lab:
   Absolute neutrophil count (ANC) ≥1.5×10⁹/L. Granulocyte-colony stimulating factor (G-CSF) cannot be administered within 2 weeks (14 days) prior to enrolment.
   Platelet count ≥100×10⁹/L.
   Hemoglobin ≥9.0 g/dL. No transfusions are allowed within 2 weeks (14 days) prior to enrolment.
   Serum creatinine ≤1.5 times upper limit of normal (ULN). Negative dip stick for proteinuria or if proteinuria ≥2+, then additional 24 h urine collection <1 g protein/24 h.
   Total bilirubin within ULN (except for subjects with Gilbert's syndrome, who must have a total bilirubin <3 mg/dL).
   Alanine aminotransferase (ALT)/aspartate aminotransferase (AST)≤3.0×ULN; if the liver has tumor involvement, AST and ALT ≤5.0×ULN are acceptable.
   An adequate coagulation function as defined by international normalized ratio (INR) ≤1.5×ULN or prothrombin time ≤1.5×ULN, and partial thromboplastin time ≤1.5×ULN (unless receiving anticoagulant therapy). Subjects receiving phenprocoumon are recommended to switch to low molecular weight heparin and should have achieved stable coagulation status prior to the first dose of study treatment.
11. Tubular excretion rate (TER) by Mercaptoacetyltriglycin (MAG-3)-clearance ≥TERLoLi (TERLoLi=70% TERNorm)
12. Eastern Cooperative Oncology Group (ECOG) performance status of 0 to 2.
13. At least 3 months' life expectancy in the investigator's assessment.

### III. Exclusion Criteria

1. Progressive disease under previous treatment with anthracyclines.
2. Subjects already enrolled in any clinical study involving an investigational product or medical device or have participated within the past 30 days in a clinical trial involving an investigational product or medical device.
3. History of another primary malignancy, with the exception of:
   curatively treated non-melanomatous skin cancer
   curatively treated cervical carcinoma in situ
   non-metastatic prostate cancer, or
   other primary non-hematologic malignancies that had been treated with curative intent, no known active disease, and no treatment administered during the last 3 years prior to enrolment, that the investigator agrees will not affect the interpretation of study results or would be unsuitable for participation in the study.
4. Active fungal, bacterial and/or known viral infection including human immunodeficiency virus or viral (A, B, or C) hepatitis.
5. Resting heart rate of >100 bpm.
6. Uncontrolled intercurrent illness including, but not limited to, an ongoing/active infection requiring parenteral antibiotics.
7. Have a serious cardiac condition, such as:
   unstable angina pectoris
   angioplasty, cardiac stenting or myocardial infarction within 6 months of enrolment
   valvulopathy that is severe, moderate, or deemed clinically significant
   arrhythmias that are symptomatic or require treatment.
9. Have a QTcF interval of >450 msec for males and >470 msec for females on screening electrocardiogram (ECG) utilizing Fridericia's correction.
10. Psychiatric illness or social situation that would limit compliance with study requirements.
11. Any planned or required major surgery during the course of the study.
12. Pregnant or breastfeeding female.

### IV. Storage and Preparation of DPPG₂-TSL-DOX

Study medication is doxorubicin (DOX) as its liposomal formulation (DPPG₂-TSL-DOX) in a cryoprotectant-containing buffered aqueous solution. Membrane bilayer of liposome is composed of the phospholipid excipients DPPC, DSPC and DPPG₂ with a molar ratio of 50:20:30, respectively. DOX is encapsulated as a crystal inside the TSL. The solution is slightly hyperosmolar (˜390 mOsmol/kg) compared to physiological solutions.

### IV.1. Storage of DPPG₂-TSL-DOX:

DPPG₂-TSL-DOX is delivered in vials (fill volume of approximately 20 ml) and stored at −20° C.+/−5° C. in the original package in order to protect from light. Temperature monitoring is required at the storage locations to ensure that the study medication is maintained within an established temperature range.

### IV.2. Preparation of DPPG₂-TSL-DOX:

DPPG₂-TSL-DOX is thawed at no more than 30° C. and diluted in a commercially available clinical infusion carrier (0.9% NaCl or 5% glucose) to an end volume of 250 mL, according to the defined clinical dose steps and subject body surface area. This dilution is performed as a reconstitution at the clinical site, immediately prior to administration of the diluted dosing solution to the subject by intravenous infusion.

### V. Treatment and Schedule of Administration of DPPG₂-TSL-DOX

### V.1 Schedule

Study treatment is administered every 3 weeks (cycle q3w) with an allowed time window of plus/minus 3 days due to weekend, holidays or other unforeseen circumstances and will not be considered as protocol violation. The actual doses of DPPG₂-TSL-DOX to be administered is determined by calculating the subject's body surface area at the beginning of each cycle.

The administration of DPPG₂-TSL-DOX is obligatorily performed via central venous access either by using an implantable port system (e.g., port-a-cath) or a central venous catheter in this study.
The starting dose of DPPG₂-TSL-DOX (dose level 1) is 20 mg/m².
The dose is escalated per dosing level as follows: dose level 2:40 mg/m², dose level 3:50 mg/m².
In all cycles, DPPG₂-TSL-DOX is applied by continuous infusion over a time period of approximately 30 minutes (250 ml/30 min). Infusion rate is approximately 8.3 mL/min and must not be exceeded.
Cycle 1 of any DPPG₂-TSL-DOX-dose level: DPPG₂-TSL-DOX-application is performed without RHT for safety precaution.
Cycle 2-6 of any DPPG₂-TSL-DOX-dose level: DPPG₂-TSL-DOX is applied in parallel with RHT as follows: RHT pre-heating is performed until target temperature of the tumor area (41.5° C. to ≤44° C.) is reached or for a maximum of 30 minutes (heating-up time). DPPG₂-TSL-DOX infusion will start if one of these requirements are fulfilled. In parallel to the start of DPPG₂-TSL-DOX infusion, therapeutic RHT will start. DPPG₂-TSL-DOX infusion is applied over 30 minutes. Therapeutic RHT is applied for 60 minutes.
Subjects whose prior cumulative dose of DOX exceeds 300 mg/m² will receive dexrazoxane for cardioprotection. Dexrazoxane will be administered at a dose equal to 10 times the DPPG₂-TSL-DOX dose (mg/m²) is administered by a short 15 minute infusion, approximately 30 minutes prior to DPPG₂-TSL-DOX infusion administration if subjects prior cumulative DOX dose is >300 mg/m².

In case of subject's weight increase or decrease of less than 10%, the DPPG₂-TSL-DOX dose will not need to be recalculated. A variance of +5% of the calculated dose is permitted for DPPG₂-TSL-DOX dose administration.

### V.2. Regional Hyperthermia

RHT treatment of the tumor area and the surrounding tissue is applied in cycles 2-6 at ≥41.5° C. to ≤44° C. for a total of 60 minutes in parallel with DPPG₂-TSL-DOX infusion as follows:
RHT pre-heating: RHT treatment is started 30 minutes prior to start of DPPG₂-TSL-DOX infusion to achieve appropriate warming of the tumor area.
Therapeutic RHT: RHT is continued for another 60 minutes (i.e., 30 minutes during DPPG₂-TSL-DOX infusion and 30 minutes following DPPG₂-TSL-DOX infusion).

### V.2.1 Application of RHT Treatment

RHT will be applied by either the Pyrexar BSD-2000@, BSD-2000/3D MR® (frequency 60-220 MHz, 0-2000 Watts) or Pyrexar BSD-500® devices (950 MHz, 0-250 Watts). Phase and amplitude steering is permitted in order to focus the heating field also on more eccentric tumors. The operational details, including subject positioning and the coupling at the body surface by means of the water bolus system, will be performed according to the ESHO quality guidelines for regional deep hyperthermia (Bruggmoser et al. 2011; Bruggmoser et al. 2012). The choice of the applicators depends on tumor location and body geometry.

By definition, the effective RHT treatment time of 60 minutes is calculated from the time when the temperature of ≥41.5° C. at any location in the tumor tissue is achieved or for a maximum of 30 minutes (heating-up time). In the initial RHT pre-heating phase (30 minutes) the power increase of the high-frequency generators is adapted to the corresponding increase of the measured temperatures in the tumor and the normal tissue. If the temperature exceeds 43.0° C. in the adjacent normal tissue, the technical parameters should be changed, otherwise the applied power is switched off or reduced until acceptable temperatures are reached. Systemic temperature must be measured regularly during hyperthermia and special attention must be paid to temperatures above 37.5° C. Cooling procedures and measurements to maintain the blood or central venous pressure should be initiated. Besides the objective criteria determined by temperature measurements, the treatment is discontinued if the subject complains about severe subjective symptoms (e.g. bolus pressure, power or position related pain) especially in the initial RHT pre-heating phase. The treatment is only continued if the cause for these symptoms can be eliminated by changing the high-frequency field or new positioning of the subject. Bladder cooling by subjects with tumors in the abdomen, pelvis or thighs is indicated.

### V.2.2 Thermometry

Invasive thermometry with intratumoral catheters is not mandatory for participation in the trial but can be performed depending on the decision of the investigator. However, for thermometry, closed-tip catheters will be placed before treatment under intermittent CT fluoroscopy guidance (Strobl et al. 2016). The catheter location and the spatial relation of the catheters to the tumor and the normal tissue will be documented by CT/MRI before starting the hyperthermia treatment.

Temperatures are measured with calibrated Bowman thermistors which allow an accurate temperature display (±0.1° C.) without interference with the radiofrequency field. The temperatures are measured at the top of the thermistors. Single Bowman thermistors are inserted into the lumen of each catheter at the time of RHT and their ends are connected to the automatic transportation system of the Pyrexar equipment. Thereby the location of all temperature probes in the catheters can be changed simultaneously. The temperatures can be measured along the catheter axis at fixed intervals (0.5 or 1.0 cm) during the RHT treatment. This mapping procedure is repeated every five minutes in order to monitor the temporal changes of this temperature distribution in the tumor tissue and in the adjacent normal tissue during the treatment. Immediately prior to each RHT treatment additional catheters may be inserted (e.g. into the rectum and bladder) to allow continuous temperature measurement in these organs. Skin temperatures should be monitored at different areas within the field of the applicator. The systemic temperature can be intermittently measured within an intra-oral probe.

### V.2.3 Thermal Dosimetric Analysis

Because of the non-uniformity of heating typically produced in tumors treated with RHT, methods of consistently characterizing non-uniform temperature distributions have been developed so that descriptors of the temperature distribution can be used in appropriate statistical analysis to be related to the outcome of therapy. For deep-seated tumors, most recent reports made use of the frequency temperature distribution of temperatures within the tumor. The time-averaged temperatures achieved in 20%, 50%, and related descriptors of the distribution such as T90 (temperature exceeded by 90% of the temperatures all measured throughout a tumor sites were determined from each RHT treatment within tumor). These values (° C.) from all RHT treatments of each subject were expressed as T20, T50 (temperature exceeded by 50% of the temperatures measured throughout a treatment within tumor), and T20 (temperature exceeded by 20% of the temperatures measured throughout a treatment within tumor). T90, respectively.

For subjects with moderate to high-grade STS, the cumulative minutes of treatment for specific T50 and T90 temperatures were good predictors of the extent of necrosis in the corresponding specimen (Leopold et al. 1992). The frequency distribution descriptors correlated more strongly with outcome than did minimum temperature. Similarly, in subjects with superficial tumors (Leopold et al. 1992). The frequency distribution descriptors correlated more strongly with the outcome than did the minimum temperature. Similarly, in subjects with superficial tumors, the cumulative duration of treatment for specific T90 temperatures was a statistically significant correlate with complete response rates, whereas minimum temperature was not (Leopold et al. 1993)

To extend the preceding concepts of thermal dosimetry, one can utilize a thermal iso-effective dose formula (Oleson et al. 1993) and convert the time/temperature records of treatment into equivalent minutes for T90 equals 40.5° C., for T50 equals 41.5° C., and T20 equals 42.5° C. in subjects with STSs. This analysis will allow determining the treatment outcome in relation to temperature, time, and thermal iso-effective dose. Therefore, the T90, T50 and T20 parameters for each RHT treatment as well as the cumulative minutes for specific T90, T50 and T20 temperatures of all RHT treatments will be calculated.

### V.3. Premedication

### V.3.1. Antihistamines and Dexamethasone

IRRs, including anaphylactoid reactions, were reported in clinical trials with liposomal chemotherapies (see 6.9.1.3). Section 6.9.3).

Therefore, premedication of all subjects with an i.v. H1 antagonist (e.g., diphenhydramine, clemastine), H2 antagonist (e.g., famotidine, cimetidine) and dexamethasone (20 mg dexamethasone or equivalent medications) intravenously 30-60 minutes prior to any DPPG₂-TSL-DOX dose must be performed. In addition, the subject will receive 8 mg Dexamethasone per os on the day before DPPG₂-TSL-DOX infusion.

Premedication with additional agents may be provided at the investigator discretion.

### 6.5.3. Antiemetic Premedication

Antiemetic premedication with a i.v. 5-HT3 receptor antagonist (type per investigator discretion and local standards-of-care) should be administered according to local standards.

### V.4. Concomitant Medication

### V.4.1. Hematopoetic Growth Factors

Primary prophylaxis with G-CSF is not required during treatment with DPPG₂-TSL-DOX in general, but only in subjects with high-risk clinical features that predispose them to increased complications from prolonged neutropenia as per ASCO guidelines (Aapro et al. 2017) and ESMO guidelines (Klastersky et al. 2016). Secondary prophylaxis with G-CSF is obligatory if any of the following criteria is met:
Occurrence of febrile neutropenia or neutropenic infection in the previous cycle
Occurrence of neutropenia ≥grade 3 (NCI-CTCAE version 5.0) in the previous cycle
Second time treatment delay >14 days due to leucopenia and/or neutropenia required
If hematologic toxicities qualifying for a DLT occur in cycle 1 or cycle 2 no further treatment with DPPG₂-TSL-DOX is allowed.

### VI. Restrictions and Precautions

DOX and other anthracyclines can cause cardiotoxicity. The risk of toxicity rises with increasing cumulative doses of those medicinal products and is higher in individuals with a history of cardiomyopathy, or mediastinal irradiation or pre-existing cardiac disease (Caelyx® SmPC). The evaluation of left ventricular function by echocardiography is considered mandatory before each administration of DPPG₂-TSL-DOX.

Plasma levels of DOX and its metabolite, doxorubicinol, may be increased when DOX is administered with cyclosporin, verapamil, paclitaxel or other agents that inhibit P-glycoprotein (P-Gp). Those substances should be recorded in the eCRF administered with special attention only.

### VII. Efficacy Evaluations/Criteria

Tumor response is determined radiologically after cycle 3 and cycle 6, with the exception of additional computed tomography (CT) or magnetic resonance imaging (MRI) imaging if the subject is clinically worsening based on the Response Evaluation Criteria in Solid Tumors RECIST version 1.1 and modified computed tomography (CT) Response Evaluation Criteria (based on Choi et al. 2007).

### VIII. Safety Evaluations and DLTs

Adverse events (AEs) and DLTs are graded using the National Cancer Institute Common Terminology Criteria for Adverse Events (NCI-CTCAE) version 5.0. AEs and DLTs are assessed at each study visit.

DLTs are the following AEs graded according to NCI-CTCAE Ver. 5.0, which the investigator or the sponsor is considering to be related to the study treatment.

### In Treatment Cycle 1 or 2:

Grade 3 or 4 febrile neutropenia or sepsis.
Grade 4 neutropenia lasting 7 days or longer.
Grade 4 thrombocytopenia, or grade 3 thrombocytopenia complicated by hemorrhage.
Non-hematologic grade ≥3 toxicity, except for disorders that can be controlled with optimal medical management within 48 hours or clinically non-significant laboratory abnormalities (such as nausea, vomiting, transient electrolyte abnormalities or diarrhea).
Renal related toxicities ≥grade 3

### In Treatment Cycle 3:

Renal related toxicities ≥grade 3

In addition to the second and routine safety monitoring (e.g., ECG, vital signs, hematology, clinical chemistry, coagulation laboratory tests), the third subject of following safety parameters for monitoring especially the given dose level have both completed cycle 1 and 2 with no observed DLTs, the next dose level can be opened after Data Safety Monitoring Board approval.

The study escalation (to 50 mg/m², dose level 3) continues as described:
kidney function are performed due to nephrotoxicity observed in pre-clinical studies:
Renal function monitoring: MAG-3-clearance
Serum: creatinine, cystatin C (CysC), estimated glomerular filtration rate (eGFR), blood urea nitrogen (BUN), magnesium (Mg), calcium (Ca), sodium (Na), potassium (K), phosphate
Urine: pH, total protein, glucose, ketones, blood, leukocytes (urine sticks), albumin, alpha 1-microglobulin (extra urinalysis).

### IX. Pharmocokinetcs

Main analytical methods for PK analysis (plasma samples) include:
Quantification of total DOX
Quantification of non-liposomal DOX
Quantification of liposomal encapsulated DOX
Quantification of the phospholipid excipient DPPG₂

### REFERENCES

Aapro M, Boccia R, Leonard R, Camps C, Campone M, Choquet S, Danova M, Glaspy J, Hus I, Link H, Sliwa T, Tesch H, Valero V. Refining the role of pegfilgrastim (a long-acting G-CSF) for prevention of chemotherapy-induced febrile neutropenia: consensus guidance recommendations. Support Care Cancer. 2017 November; 25 (11): 3295-3304
Aklan B, Zilles B, Paprottka P, Manz K, Pfirrmann M, Santl M, Abdel-Rahman S, Lindner L H. Regional deep hyperthermia: quantitative evaluation of predicted and direct measured temperature distributions in patients with high-risk extremity soft-tissue sarcoma. Int J Hyperthermia. 2019; 36 (1): 170-185.), Kok et al. (Kok H P, Wust P, Stauffer P R, Bardati F, van Rhoon G C, Crezee J. Current state of the art of regional hyperthermia treatment planning: a review. Radiat Oncol. 2015 Sep. 17; 10:196
Bruggmoser G, Bauchowitz S, Canters R, Crezee H, Ehmann M, Gellermann J, Lamprecht U, Lomax N, Messmer M B, Ott O, Abdel-Rahman S, Schmidt M, Sauer R, Thomsen A, Wessalowski R, van Rhoon G; Atzelsberg Research Group; European Society for Hyperthermic Oncology. Guideline for the clinical application, documentation and analysis of clinical studies for regional deep hyperthermia: quality management in regional deep hyperthermia. Strahlenther Onkol. 2012 September; 188 Suppl 2:198-211
Bruggmoser G, Bauchowitz S, Canters R, Crezee H, Ehmann M, Gellermann J, Lamprecht U, Lomax N, Messmer M B, Ott O, Abdel-Rahman S, Sauer R, Schmidt M, Thomsen A, Wessalowski R, van Rhoon G; ESHO Technical Committee in the Interdisciplinary Working Group Hyperthermia (IAH) in the German Cancer Society. Quality assurance for clinical studies in regional deep hyperthermia. Strahlenther Onkol. 2011 October; 187 (10): 605-10
Carter D L, MacFall J R, Clegg S T, Wan X, Prescott D M, Charles H C, Samulski T V. Magnetic resonance thermometry during hyperthermia for human high-grade sarcoma. Int J Radiat Oncol Biol Phys. 1998 Mar. 1; 40 (4): 815-22
Choi H, Charnsangavej C, Faria S C, Macapinlac H A, Burgess M A, Patel S R, Chen L L, Podoloff D A, Benjamin R S. Correlation of computed tomography and positron emission tomography in patients with metastatic gastrointestinal stromal tumor treated at a single institution with imatinib mesylate: proposal of new computed tomography response criteria. J Clin Oncol. 2007 May 1; 25 (13): 1753-9
Drizdal T, Paulides M M, van Holthe N, van Rhoon G C. Hyperthermia treatment planning guided applicator selection for sub-superficial head and neck tumors heating. Int J Hyperthermia. 2018 September; 34 (6): 704-713.
Eisenhauer E A, Therasse P, Bogaerts J, Schwartz L H, Sargent D, Ford R, Dancey J, Arbuck S, Gwyther S, Mooney M, Rubinstein L, Shankar L, Dodd L, Kaplan R, Lacombe D, Verweij J. New response evaluation criteria in solid tumours: revised RECIST guideline (version 1.1). Eur J Cancer. 2009 January; 45 (2): 228-47
Hossann M, Wang T, Wiggenhorn M, Schmidt R, Zengerle A, Winter G, Eibl H, Peller M, Reiser M, Issels R D, Lindner L H. Size of thermosensitive liposomes influences content release. J Control Release. 2010 Nov. 1; 147 (3): 436-43
Hossann M, Wiggenhorn M, Schwerdt A, Wachholz K, Teichert N, Eibl H, Issels R D, Lindner L H. In vitro stability and content release properties of phosphatidylglyceroglycerol containing thermosensitive liposomes. Biochim Biophys Acta. 2007 October; 1768 (10): 2491-9
Hossann H, Hirschberger J, Schmidt R, Baumgartner C, Zimmermann K, Baer S, Ratzlaff C, Peller M, Troedson K, Limmer S, Bruhschwein A, Dorfelt R, Kreutzmann N, Wess G, Knösel T, Schagon O, Fischer J, Grüll H, Willerding L, Schmidt M, Meyer-Lindenberg A, Issels R D, Schwaiger M, Eggermont A M, Ten Hagen T L, Lindner L H. A heat-activated drug delivery platform based on phosphatidyl-(oligo)-glycerol nanocarrier for effective cancer treatment, Advanced NanoBiomed Research. 2021; 1 (6): 2000089.
Issels R D, Lindner L H, Verweij J, Wessalowski R, Reichardt P, Wust P, Ghadjar P, Hohenberger P, Angele M, Salat C, Vujaskovic Z, Daugaard S, Mella O, Mansmann U, Dürr HR, Knösel T, Abdel-Rahman S, Schmidt M, Hiddemann W, Jauch K W, Belka C, Gronchi A; European Organization for the Research and Treatment of Cancer-Soft Tissue and Bone Sarcoma Group and the European Society for Hyperthermic Oncology. Effect of Neoadjuvant Chemotherapy Plus Regional Hyperthermia on Long-term Outcomes Among Patients With Localized High-Risk Soft Tissue Sarcoma: The EORTC 62961-ESHO 95 Randomized Clinical Trial. JAMA Oncol. 2018 Apr. 1; 4 (4): 483-492.
Klastersky J, de Naurois J, Rolston K, Rapoport B, Maschmeyer G, Aapro M, Herrstedt J; ESMO Guidelines Committee. Management of febrile neutropaenia: ESMO Clinical Practice Guidelines. Ann Oncol. 2016 September; 27 (suppl 5): v111-v118
Kok H P, Wust P, Stauffer P R, Bardati F, van Rhoon G C, Crezee J. Current state of the art of regional hyperthermia treatment planning: a review. Radiat Oncol. 2015 Sep. 17; 10:196
Leopold K A, Dewhirst M, Samulski T, Harrelson J, Tucker J A, George S L, Dodge R K, Grant W, Clegg S, Prosnitz L R, et al. Relationships among tumor temperature, treatment time, and histopathological outcome using preoperative hyperthermia with radiation in soft tissue sarcomas. Int J Radiat Oncol Biol Phys. 1992; 22 (5): 989-98
Leopold K A, Dewhirst M W, Samulski T V, Dodge R K, George S L, Blivin J L, Prosnitz L R, Oleson J R. Cumulative minutes with T90 greater than Tempindex is predictive of response of superficial malignancies to hyperthermia and radiation. Int J Radiat Oncol Biol Phys. 1993 Apr. 2; 25 (5): 841-7
Lindner L H, Eichhorn M E, Eibl H, Teichert N, Schmitt-Sody M, Issels R D, Dellian M. Novel temperature-sensitive liposomes with prolonged circulation time. Clin Cancer Res. 2004 Mar. 15; 10 (6): 2168-78
Lyon P C, Gray M D, Mannaris C, Folkes L K, Stratford M, Campo L, Chung D Y F, Scott S, Anderson M, Goldin R, Carlisle R, Wu F, Middleton M R, Gleeson F V, Coussios C C. Safety and feasibility of ultrasound-triggered targeted drug delivery of doxorubicin from thermosensitive liposomes in liver tumours (TARDOX): a single-centre, open-label, phase 1 trial. Lancet Oncol. 2018 August; 19 (8): 1027-1039
Needham, G. Anyarambhatla, G. Kong, M. W. Dewhirst, A new temperature-sensitive liposome for use with mild hyperthermia: characterization and testing in a human tumor xenograft model, Cancer Res 60 (2000) 1197-1201
Oleson J R, Samulski T V, Leopold K A, Clegg S T, Dewhirst M W, Dodge R K, George S L. Sensitivity of hyperthermia trial outcomes to temperature and time: implications for thermal goals of treatment. Int J Radiat Oncol Biol Phys. 1993 Jan. 15; 25 (2): 289-97
Paulides M M, Dobsicek Trefna H, Curto S, Rodrigues D B. Recent technological advancements in radiofrequency- and microwave-mediated hyperthermia for enhancing drug delivery. Adv Drug Deliv Rev. 2020; 163-164:3-18
Rivankar S. An overview of doxorubicin formulations in cancer therapy. J Cancer Res Ther. 2014 October-December; 10(4):853-8
Strobl F F, Azam H, Schwarz J B, Paprottka P M, Geith T, Abdel-Rahman S, Zilles B, Lindner L H, Reiser M F, Trumm C G. CT fluoroscopy-guided closed-tip catheter placement before regional hyperthermia treatment of soft tissue sarcomas: 5-Year experience in 35 consecutive patients. Int J Hyperthermia. 2016; 32 (2): 151-8
Tak W Y, Lin S M, Wang Y, Zheng J, Vecchione A, Park S Y, Chen M H, Wong S, Xu R, Peng C Y, Chiou Y Y, Huang G T, Cai J, Abdullah B J J, Lee J S, Lee J Y, Choi J Y, Gopez-Cervantes J, Sherman M, Finn R S, Omata M, O'Neal M, Makris L, Borys N, Poon R, Lencioni R. Phase III HEAT Study Adding Lyso-Thermosensitive Liposomal Doxorubicin to Radiofrequency Ablation in Patients with Unresectable Hepatocellular Carcinoma Lesions. Clin Cancer Res. 2018 Jan. 1; 24 (1): 73-83
van Valenberg F J P, Brummelhuis I S G, Lindner L H, Kuhnle F, Wedmann B, Schweizer P, Hossann M, Witjes J A, Oosterwijk E. DPPG₂-Based Thermosensitive Liposomes with Encapsulated Doxorubicin Combined with Hyperthermia Lead to Higher Doxorubicin Concentrations in the Bladder Compared to Conventional Application in Pigs: A Rationale for the Treatment of Muscle-Invasive Bladder Cancer. Int J Nanomedicine. 2021 Jan. 7; 16:75-88.
Waterhouse D N, Tardi P G, Mayer L D, Bally M B. A comparison of liposomal formulations of doxorubicin with drug administered in free form: changing toxicity profiles. Drug Saf. 2001; 24 (12): 903-20.
Willerding L, Limmer S, Hossann M, Zengerle A, Wachholz K, Ten Hagen T L, Koning G A, Sroka R, Lindner L H, Peller M. Method of hyperthermia and tumor size influence effectiveness of doxorubicin release from thermosensitive liposomes in experimental tumors. J Control Release. 2016 Jan. 28; 222:47-55
Zagar T M, Vujaskovic Z, Formenti S, Rugo H, Muggia F, O'Connor B, Myerson R, Stauffer P, Hsu I C, Diederich C, Straube W, Boss M K, Boico A, Craciunescu O, Maccarini P, Needham D, Borys N, Blackwell K L, Dewhirst M W. Two phase I dose-escalation/pharmacokinetics studies of low temperature liposomal doxorubicin (LTLD) and mild local hyperthermia in heavily pretreated patients with local regionally recurrent breast cancer. Int J Hyperthermia. 2014 August; 30 (5): 285-94
Zhu L, Altman M B, Laszlo A, Straube W, Zoberi I, Hallahan D E, Chen H. Ultrasound Hyperthermia Technology for Radiosensitization. Ultrasound Med Biol. 2019 May; 45 (5): 1025-1043

1.-16. (canceled)

17. A method of treating cancer in a human subject, the method comprising administering a liposome, comprising doxorubicin or a pharmaceutically acceptable salt or derivative thereof to the subject, wherein the liposome is administered to the subject at a dose of about 20 to about 80 mg/m².

18. The method according to claim 17, wherein the liposome is a thermosensitive liposome.

19. The method according to claim 17, wherein the liposome does not comprise a lysolipid in the bilayer.

20. The method according to claim 17, wherein the liposome comprises 1,2-dipalmitoyl-sn-glycero-3-phosphooligoglycerol (DPPGₙ), preferably 1,2-dipalmitoyl-sn-glycero-3-phosphodiglycerol (DPPG₂), in the bilayer.

21. The method according to claim 17, wherein
a) prior to the administration of the liposome, or
b) after or concomitantly with the start of the administration of the liposome,
a heat treatment is administered until a tumor temperature of from about 38 to about 45° C.

22. The method according to claim 17, wherein a heat treatment is administered during the administration of the liposome which increases and/or maintains the tumor temperature to/at from about 38° C. to about 45° C.

23. The method according to claim 17, wherein a heat treatment is administered after the administration of the liposome is terminated which maintains the tumor temperature at from about 38° C. to about 45° C.

24. The method according to claim 21, wherein the heat treatment is administered for at least about 10 min prior to the administration of the liposome and/or wherein the heat treatment is administered until at least about 10 min after termination of the administration of the liposome.

25. The method according to claim 21, wherein the heat treatment is administered not longer than 30 min prior to the administration of the liposome and/or wherein the heat treatment is administered until about 45 after the termination of the administration of the liposome.

26. The method according to claim 17, wherein the cancer is a soft tissue sarcoma, preferably undifferentiated pleomorphic sarcoma (UPS), liposarcoma (well differentiated liposarcoma, dedifferentiated liposarcoma, myxoid liposarcoma, pleomorphic liposarcoma), leiomyosarcoma, synovial sarcoma, angiosarcoma, epithelioid sarcoma, malignant peripheral nerve sheath tumor (MPNST), rhabdomyosarcoma (alveolar rhabdomyosarcoma, embryonal rhabdomyosarcoma, pleomorphic rhabdomyosarcoma), solitary fibrous tumor, myxofibrosarcoma, fibrosarcoma, uterine sarcoma (uterine leiomyosarcoma, endometrial stromal sarcoma), desmoplastic small round cell sarcoma (DSRCT), desmoids, kaposi sarcoma or bone sarcoma, preferably Ewing sarcoma, osteosarcoma or chondrosarcoma.

27. The method according to claim 17, wherein the cancer is a locally advanced or metastatic.

28. The method according to claim 17, wherein the treated tumor of said cancer has a volume of at least about 30 cm³, and/or wherein the treated tumor of said cancer has a diameter of at least 4 cm.

29. The method according to claim 21, wherein the heat treatment is administered in form of local hyperthermia, regional hyperthermia, or superficial hyperthermia, preferably as regional deep hyperthermia, and/or
wherein the heat treatment is administered in form of ultrasound induced hyperthermia, microwave frequency induced hyperthermia or hyperthermic perfusion,
wherein optionally the ultrasound or microwave frequency induced hyperthermia is administered by external administration.

30. The method according to claim 17, wherein the liposome is administered in a treatment cycle every 7 to 28 days, preferably every 21 days.

31. The method according to claim 17, wherein a premedication, preferably with a compound selected from the group comprising steroids, dexrazoxane, and/or H1-receptor antagonist, is administered prior to the administration of the liposome.

32. The method according to claim 20, wherein the concentration of said DPPG₂ in said bilayer is from 15 mol % up to 55 mol %, preferably from 25 mol % up to 35 mol %, and/or
wherein said 1,2-dipalmitoyl-sn-glycero-3-phosphodiglycerol (DPPG₂) is represented by Formula (I): 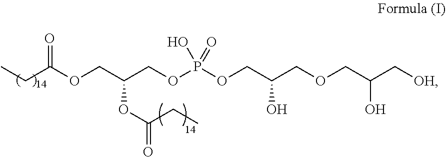
and/or
wherein said bilayer comprises 1,2-dipalmitoyl-sn-glycero-3-phosphocholine (DPPC) and/or 1,2-distearoyl-sn-glycero-3-phosphocholine (DSPC), wherein
preferably the molar concentration of 1,2-dipalmitoyl-sn-glycero-3-phosphocholine (DPPC) in said bilayer is from 30 mol % up to 70 mol %; and/or
wherein preferably the molar concentration of 1,2-distearoyl-sn-glycero-3-phosphocholine (DSPC) in said bilayer is from 5 mol % up to 35 mol %; and/or
wherein preferably the molar concentration of 1,2-dipalmitoyl-sn-glycero-3-phosphodiglycerol (DPPG₂) in said bilayer is from 15 mol % up to 55 mol %.
